(19) United States
(12) Patent Application Publication
(10) Pub. No.: US 2005/0096708 A1
(43) Pub. Date: May 5, 2005

(54) Atrial antitachycardia pacing management

(76) Inventors: Gary T. Seim, Minneapolis, MN (US); James O. Gilkerson, Stillwater, MN (US)

Correspondence Address: Crawford Maunu PLLC, Suite 390, 1270 Northland Drive, St. Paul, MN, 55120, US

(21) Appl. No.: 10/698,858

(22) Filed: Oct. 31, 2003

(51) Int. Cl. A61N 1/37

(52) U.S. Cl. 607/28; 607/14

(57) ABSTRACT

A system and method provides for managing atrial ATP therapy in response to possible atrial lead dislodgment. An impedance of an atrial lead is measured for a particular patient. The measured impedance is compared with an impedance threshold developed for the particular patient. Atrial ATP therapy delivery is disabled in response to the measured impedance deviating from the impedance threshold by a predetermined factor. The impedance threshold may be developed from one or more atrial lead impedance measurements, and may also be characterized by a mean or a median of several atrial lead impedance measurements. The predetermined factor may be characterized by a percentage change, a fixed delta change, or both a percentage change and a fixed delta change in the measured impedance relative to the impedance threshold.

### FIELD OF THE INVENTION

The present invention relates generally to implantable medical devices and, more particularly, to implantable pacemakers, cardioverter-defibrillators, resynchronizers, and other cardiac stimulation devices that provide atrial antitachycardia pacing management.

### BACKGROUND OF THE INVENTION

Implantable cardioverter-defibrillators (ICDs) have been developed that employ detection algorithms capable of recognizing and treating atrial tachycardias and atrial fibrillation. In general, ICDs are designed to treat such tachycardias with antitachycardia pacing and low-energy cardioversion shocks in conjunction with back-up defibrillation therapy. These ICDs monitor the heart rate and the onset of the arrhythmia by sensing endocardial signals and determining when the heart is in need of either cardioversion to treat a given tachycardia or of defibrillation to treat a fibrillation condition.

Certain ICDs have been designed with dual chamber sensing capabilities to detect and analyze both ventricular and atrial endocardial signals. This increase in cardiac signal input to the ICD has provided an opportunity to determine the origin and the nature of atrial and ventricular tachyarrhythmia, and to reduce the frequency of inappropriate therapy being delivered to an implant patient. However, while the combination of antitachycardia pacing with low and high energy shock delivery, as well as backup bradycardia pacing, in ICDs has expanded the number of clinical situations in which the devices may appropriately be employed, the safe delivery of such therapies depends largely on the reliability of lead positioning within the atrium and accurate detection atrial lead dislodgement over time.

### SUMMARY OF THE INVENTION

Embodiments of the present invention are directed to systems and methods for managing atrial arrhythmia therapy, such as atrial antitachycardia pacing (ATP) therapy, in a cardiac stimulation device. Embodiments of the present invention are also directed to systems and methods for detecting atrial lead dislodgement. Embodiments of the present invention are further directed to managing atrial arrhythmia therapy, such as atrial ATP therapy, in response to detection of atrial lead dislodgment.

According to one embodiment, a method of managing atrial ATP therapy in response to possible atrial lead dislodgment involves measuring an impedance of an atrial lead for a particular patient, comparing the measured impedance with an impedance threshold developed for the particular patient, and disabling atrial ATP therapy delivery in response to the measured impedance deviating from the impedance threshold by a predetermined factor. The impedance threshold may be developed from one or more atrial lead impedance measurements, and may also be characterized by a mean or a median of several atrial lead impedance measurements. The predetermined factor may be characterized by a percentage change, a fixed delta change, or both a percentage change and a fixed delta change in the measured impedance relative to the impedance threshold.

Measuring the impedance may involve delivering a pace pulse via the atrial lead and deriving the impedance measurement using the delivered pace pulse. According to another approach, measuring the impedance involves delivering a stimulus via the atrial lead and deriving the impedance measurement using the delivered stimulus, wherein the stimulus has an energy insufficient to effect atrial capture. Atrial lead impedance may be measured before and/or after detection of an atrial arrhythmic event or episode, and prior to atrial ATP therapy delivery.

According to another embodiment, a method of managing atrial ATP therapy in response to possible atrial lead dislodgment involves measuring an impedance, a capture threshold, and a sense amplitude respectively associated with an atrial lead for a particular patient. The method also involves comparing the impedance, capture threshold, and sense amplitude measurements with impedance, capture threshold, and sense amplitude limits, respectively. Atrial ATP therapy delivery is disabled in response to any of the impedance, capture threshold, and sense amplitude measurements deviating from the impedance, capture threshold, and sense amplitude limits by predetermined impedance, capture threshold, and sense amplitude factors, respectively.

The method may also involve detecting an ambiguity in the impedance, capture threshold, and sense amplitude deviations, and disabling atrial ATP therapy delivery in response to the detected ambiguity. For example, the method may further involve disabling atrial ATP therapy delivery in response to the measured impedance deviating from the impedance limit by the predetermined factor irrespective of a lack of ambiguity relative to the capture threshold and sense amplitude deviations. By way of further example, disabling atrial ATP therapy delivery may further involve ignoring, upon detection of an atrial arrhythmia, the capture threshold and sense amplitude deviations, and disabling atrial ATP therapy in response only to the measured impedance deviating from the impedance limit by the predetermined factor.

In accordance with another embodiment, an apparatus for managing atrial ATP therapy in response to possible atrial lead dislodgment includes an implantable housing and detection circuitry provided in the housing. Energy delivery circuitry is also provided in the housing. A lead system, comprising at least an atrial lead, is coupled to the detection and energy delivery circuitry, respectively. A control system is provided in the housing and coupled to memory. An impedance threshold developed for a particular patient is stored in the memory. The control system measures an impedance of the atrial lead for the particular patient and compares the measured-impedance with the impedance threshold. The control system disables atrial ATP therapy delivery in response to the measured impedance deviating from the impedance threshold by a predetermined factor.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a depiction of an implantable medical device with which the atrial antitachycardia therapy management methodologies of the present invention may be practiced;

FIG. 2 is a block diagram of several components housed in the implantable medical device of FIG. 1;

FIG. 3 is a flow diagram depicting several processes of an atrial ATP management methodology implemented by a cardiac stimulation device in accordance with an embodiment of the present invention;

FIG. 4 is a flow diagram depicting several processes of an atrial ATP management methodology implemented by a cardiac stimulation device in accordance with another embodiment of the present invention;

FIG. 5 is a flow diagram depicting several processes of an atrial ATP management methodology implemented by a cardiac stimulation device in accordance with a further embodiment of the present invention; and

FIG. 6 is a flow diagram depicting several processes of an atrial ATP management methodology implemented by a cardiac stimulation device in accordance with yet another embodiment of the present invention.

While the invention is amenable to various modifications and alternative forms, specifics thereof have been shown by way of example in the drawings and will be described in detail hereinbelow. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings which form a part hereof, and in which is shown by way of illustration, various embodiments in which the invention may be practiced. It is to be understood that other embodiments may be utilized, and structural and functional changes may be made without departing from the scope of the present invention.

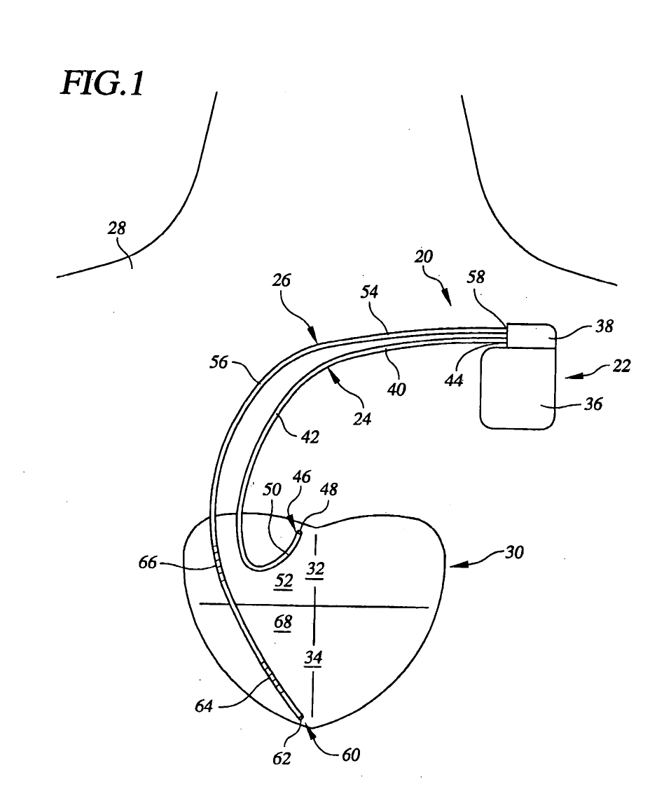

Referring now to the figures, and more particularly to FIG. 1, there is shown a body implantable system 20 that represents one of several types of systems with which the atrial antitachycardia therapy management methodologies of the present invention may be practiced. For example, the implantable pulse generator 22 may be representative of all or part of a pacemaker, defibrillator, cardioverter, cardiac monitor, or re-synchronization device (e.g., multichamber or multisite device). Accordingly, the atrial antitachycardia therapy management methodologies of the present invention may be practiced in a wide variety of implantable medical devices that sense cardiac activity.

The body implantable system 20 is shown to include an implantable pulse generator 22 coupled to an atrial lead 24 and a ventricular lead 26. The system 20 may also include endocardial pacing and cardioversion/defibrillation leads (not shown) that are advanced into the coronary sinus and coronary veins to locate the distal electrode(s) adjacent to the left ventricle or the left atrium.

The system 20, as shown in FIG. 1, is implanted in a human body 28 with portions of the atrial and ventricular leads 24 and 26 inserted into a heart 30 to detect and analyze electric cardiac signals produced by both the atria 32 and the ventricles 34 of the heart 30. The atrial and ventricular leads 24 and 26 also provide electrical energy to the heart 30 under certain predetermined conditions to treat various types of cardiac arrhythmia, including, for example, atrial and ventricular tachycardias, and atrial and ventricular fibrillation of the heart 30.

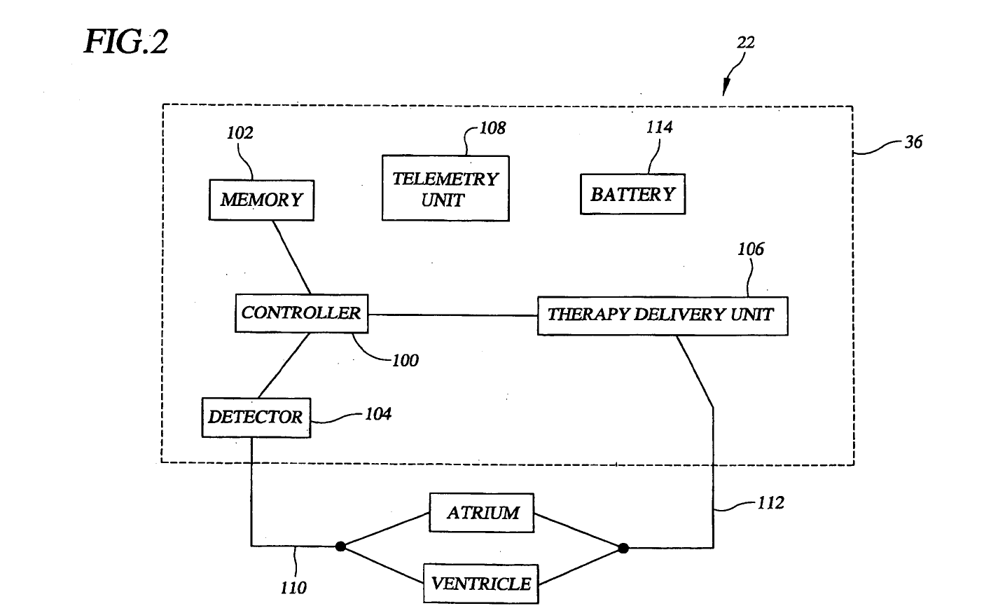

A block diagram of the implantable pulse generator 22 electronics is provided in FIG. 2. The implantable pulse generator 22 includes a housing 36 which contains, among other components, a controller 100 and memory 102, which typically includes read only memory (ROM) and random access memory (RAM). Pulse generator 22 further includes a detector 104, which includes atrial and ventricular sense amplifiers (not shown), a therapy delivery unit 106, and a telemetry unit 108. The electronic components of the pulse generator 22 are interconnected by way of a bus connection (not shown).

Power to the implantable pulse generator 22 is supplied by an electrochemical battery 114 which is contained within the implantable pulse generator housing 36. The implantable pulse generator 22 is interrogated and programmed via bi-directional radio frequency telemetry through cooperative operation between the telemetry unit 108 and an external programmer in a manner known in the art.

The atrial antitachycardia therapy management methodologies implemented by system 20 are embodied in one or more algorithms as firmware within memory 102, and are executed by the controller 100. The detector 104 is also connected to the controller 100, and contains a plurality of electrical connections 110 coupled to the atrial and ventricular sense amplifiers. The outputs of the sense amplifiers are connected to the controller 100, such that atrial and ventricular signals received through the detector 104 are analyzed by the algorithms implemented within the controller 100. The controller 100 is also coupled to the therapy delivery unit 106, which controls the delivery of electrical energy to the heart 30 through a plurality of electrical output connections 112 to affect the sinus rhythm of the heart 30 under certain combinations of atrial 32 and ventricular 34 conditions.

Referring again to FIG. 1, a connector block 38 is mounted on the implantable pulse generator 22. The connector block 38 has two connector ports for coupling the atrial lead 24 and the ventricular lead 26 to the detector 104 and the therapy delivery unit 106 of the implantable pulse generator 22. Additional connector ports can be added to the connector block 38, as in the case of configurations having three or more ports as is known in the art. Alternatively, the connector block 38 can be provided with one connector port for coupling an implantable transvenous lead to the implantable pulse generator 22. It is understood that atrial and ventricular sensing and pacing/defibrillating functions may be accomplished using a single lead system employing atrial and ventricular conductors/electrodes, rather than by use of the dual lead system shown in FIG. 1.

In general, the electrical activity in the heart 30 is sensed, and therapies are delivered to the heart 30, through at least one transvenous pacing/defibrillation lead connected to the implantable pulse generator 22. Unipolar and/or bipolar pacing and sensing electrodes can be used in conjunction with the transvenous pacing/defibrillation lead. In the embodiment shown in FIG. 1, bipolar leads and sensing circuits are utilized for sensing both the atrial 32 and the ventricular 34 activity. Sensing atrial activity includes the determination of atrial P-waves for purposes of determining atrial intervals. Ventricular activity is monitored by sensing for the occurrence of ventricular R-waves for purposes of determining ventricular intervals. Pacing therapies to the atrium 32 or ventricle 34 are delivered to the heart 30 using these same leads.

The system 20 may also employ defibrillation electrodes which are connected to the electrical output connections 112, and serve to deliver cardioversion and defibrillation level electrical pulses to the heart 30 as determined by the programming of controller 100. The housing 36 of the system 20 may be used as an optional defibrillation electrode, where the housing 36 of the implantable pulse generator 22 is electrically connected to a cathode pole of the therapy delivery unit 106. All defibrillation electrical pulses are delivered to the heart with at least two defibrillation electrodes, or through at least one defibrillation electrode and the housing 36 of the implantable pulse generator 22. The system 20 supports a plurality of pacing regimens.

In addition to the lead configuration shown in FIG. 1, the system 20 supports several other lead configurations and types. For example, it is possible to use ventricular epicardial rate sensing, atrial endocardial bipolar pace/sensing, ventricular endocardial bipolar pace/sensing, epicardial patches, and ancillary leads in conjunction with the implantable pulse generator 22.

In the embodiment of system 20 depicted in FIG. 1, the atrial lead 24 has an elongated body 40 having a peripheral surface 42, proximal and distal ends, 44 and 46, a first atrial electrode 48, and a second atrial electrode 50 on the peripheral surface 42. The first atrial electrode 48 and the second atrial electrode 50 receive bipolar electrical cardiac signals from the right atrium chamber 52 of the heart 30, and are attached on the peripheral surface 42 of the elongated body 40.

The first atrial electrode 48 is situated at or adjacent to the distal end 46 of the elongated body 40 and is either a pacing tip electrode or a semi-annular or annular electrode partially or completely encircling the peripheral surface 42 of the elongated body 40. The second electrode 50 is an annular or semi-annular electrode encircling or partially encircling the peripheral surface 42 of the elongated body 40. The second electrode 50 is spaced longitudinally along the peripheral surface 40 from the first atrial electrode 48 and the distal end 46 of the atrial lead 24, such that when the atrial lead 24 is inserted into the right atrial chamber 52 of the heart 30, the first atrial electrode 48 is in physical contact with a portion of a wall of the right atrial chamber 52 of the heart 30 and the second electrode 50 is within the right atrium chamber 52.

Electrical conductors extend longitudinally within the elongated body 40 of the atrial lead 24 from a connection end at the proximal end 44 and make connection to the first and second atrial electrodes 48 and 50. The proximal end 44 of the atrial pacing lead 24 is attached to the connector block 38 of the implantable pulse generator 22. The connector block 38 provides electrical coupling between the contact ends of the electrical conductors of atrial lead 24 with the atrial sense amplifier of the detector 104 and the therapy delivery unit 106, such that the implantable pulse generator 22 receives bipolar signals from, and delivers bipolar pacing to, the right atrium 52 of the heart 30.

The ventricular lead 26 includes an elongated body 54 having a peripheral surface 56, proximal and distal ends, 58 and 60, and a ventricle pacing electrode 62. The ventricular lead 26 also includes a first defibrillation electrode 64 and a second defibrillation electrode 66 situated on the peripheral surface 56 of the elongated body 54. The ventricular pacing electrode 62 and the first defibrillation electrode 64 are adapted to receive electrical cardiac signals from the right ventricle chamber 68 of the heart 30, and are attached on the peripheral surface of the elongated body 54. The second defibrillation electrode 66 is spaced apart and longitudinally on the peripheral surface 56 of the ventricular lead 26. This configuration affords positioning of the ventricular lead 26 in the heart 30 with the ventricular pacing electrode 62 in the apex of the right ventricle 68, the first defibrillation electrode 64 within the right ventricle chamber of the heart, and the second defibrillation electrode 66 within the right atrium chamber or a major vein leading to the right atrium 52.

Electrical conductors extend longitudinally within the elongated body 54 of the ventricular lead 26 from a connection end at the proximal end 58 to make connection with the ventricular pacing electrode 62, the first defibrillation electrode 64, and the second defibrillation electrode 66. The proximal end 58 of the ventricular lead 26 is attached to the connector block 38 of the implantable pulse generator 22. The connector block 38 provides for electrical coupling between the contact ends of the electrical conductors of ventricular lead 26 with the ventricular sense amplifier of the detector 104 and the therapy delivery unit 106, such that the implantable pulse generator 22 receives either unipolar or bipolar signals from, and can deliver unipolar or bipolar pacing to, the right ventricle 68 and defibrillation electrical pulses to the ventricles 34 of the heart 30.

The atrial lead 24 and the ventricular lead 26 are attachable to, and separable from, the implantable pulse generator 22 to facilitate insertion of the atrial lead 24 into the heart 30. The proximal end 44 of the atrial lead 24 and the proximal end 58 of the ventricular lead 26 are adapted to seal together with the connector ports of the implantable pulse generator 22 to thereby engage the contact ends of the atrial lead 24 and the ventricular lead 26 with the plurality of electrical connections 110 and the therapy delivery unit 106 of the implantable pulse generator 22. The implantable pulse generator 22 of the system 20 is then positioned subcutaneously within the body 28.

Dual and multiple chamber cardiac stimulation devices can be implemented to provide atrial tachyarrythmia therapy, such as atrial antitachycardia pacing (ATP), to address persistent atrial arrhythmic conditions. Although such devices can effectively treat atrial arrhythmias, it is known that delivery of atrial shock therapy can cause ventricular pro-arrhythmia. However, conventional cardiac stimulation devices that provide atrial antitachycardia therapy are designed to significantly mitigate the risk of ventricular pro-arrhythmia though enablement of various features, such as R-wave synchronization and ventricular arrhythmia detection and therapy.

Notwithstanding such mitigation strategies, it is possible that atrial lead dislodgment into the ventricle can result in unintended and dangerous delivery of atrial antitachycardia therapy to the ventricle. For example, atrial lead dislodgment into the ventricle can result in sensing both atrial and ventricular depolarizations as an atrial arrhythmia. Atrial ATP, for example, would then be delivered to the ventricle, thereby inducing a true ventricular arrhythmia.

Continuing with this potential scenario, ventricular therapy would then be delivered to convert the ventricular arrhythmia. Again, the atrial and ventricular depolarizations would be sensed as an atrial arrhythmia. Atrial ATP therapy would once again be delivered creating a ventricular arrhythmia, and ventricular therapy would again be delivered. With atrial lead dislodgment, it is possible for this cycle to continue.

An atrial ATP management methodology of the present invention provides for enhanced safety against ventricular arrhythmia inadvertently induced through delivery of atrial tachyarrhythmia therapy via an atrial lead dislodged into a ventricle. In broad and general terms, an atrial ATP management methodology of the present invention monitors a parameter indicative of atrial lead condition and detects an aberrant atrial lead condition that could indicate dislodgement of the atrial lead. Detecting such an aberrant atrial lead condition is preferably based on detection of a large, unexpected change in the monitored parameter. Detecting large, unexpected changes in a monitored parameter that indicate likely or actual occurrence of atrial lead dislodgement results in disabling of atrial tachyarrhythmia therapy as a preventative measure against atrial lead induced ventricular arrhythmia.

Generally, one or more parameters associated with an atrial lead are measured for purposes of evaluating implantation integrity of the atrial lead for a particular patient. These parameters are typically selected to facilitate easy and accurate detection of atrial lead implantation integrity/dislodgment for a particular patient. Such parameters are preferably determined periodically to establish baseline values for the parameters. Large, unexpected deviations in one or more parameter measurements relative to their baseline values provide an indication that atrial lead dislodgement has likely or actually occurred, and that atrial ATP therapy is to be disabled and the cause of such deviations explored by the patient's physician.

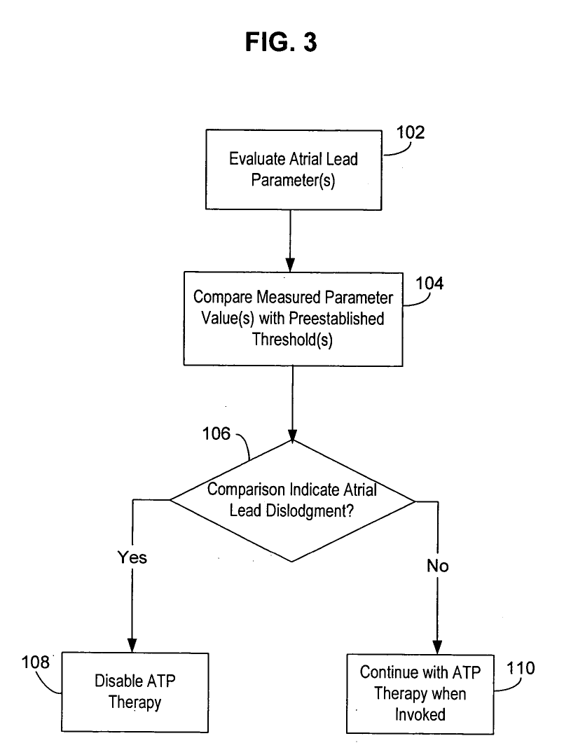

Referring now to FIG. 3, there is shown a flow diagram depicting several processes of an atrial ATP management methodology implemented by system 20 or other cardiac stimulation device in accordance with an embodiment of the present invention. According to the general methodology shown in FIG. 3, one or more atrial lead parameters are evaluated 102. Typically, measurements of pre-selected atrial lead parameters are taken and compared 104 with a pre-established threshold(s). If the comparison indicates 106 probable atrial lead dislodgment, atrial ATP therapy delivery is disabled 108. If the comparison does not indicate 106 probable atrial lead dislodgment, atrial ATP therapy delivery remains available 110 when invoked.

In general, a comparison of a pre-selected atrial lead parameter with a preestablished threshold indicates probable atrial lead dislodgment when the measured atrial lead parameter value deviates from the preestablished threshold by a sufficiently large amount (e.g., a step function change in the measured parameter). If such a sufficiently large deviation is detected, dislodgment of the atrial lead from atrial tissue is assumed, and ATP therapy delivery is disabled.

Detection of a sufficiently large deviation between a measured atrial lead parameter value and a preestablished threshold may be determined in a number of ways, such as by evaluating percentage changes, fixed delta changes, or combinations of these changes in the measured atrial lead parameter. The atrial lead parameter measurement may be characterized by a single atrial lead parameter measurement or several atrial lead parameter measurements.

Sufficiently large changes in a measured atrial lead parameter value that can trigger ATP therapy disablement are those that are significantly larger than would be expected for a given lead/electrode and patient. Such gross changes are typically appreciably larger than a tolerance associated with a given atrial lead parameter. For example, changes in excess of 25% in a measured atrial lead parameter value can trigger ATP therapy disablement. By way of further example, changes in excess of 10% beyond a tolerance range of a given atrial lead parameter may be considered a sufficiently large change that warrants disablement of ATP therapy delivery.

The preestablished threshold may be developed for a particular patient using a single atrial lead parameter measurement. A number of atrial lead parameter measurements may be taken to characterize the preestablished threshold. A mean or a median of a number of atrial lead parameter measurements may be computed and used to characterize the preestablished threshold. In another approach, the preestablished threshold may be characterized by an atrial lead parameter measurement made immediately before a currently taken atrial lead parameter measurement. In yet another approach, the preestablished threshold may be characterized by at least one atrial lead parameter measurement made a predetermined amount of time (e.g., one day) prior to a currently taken atrial lead parameter measurement.

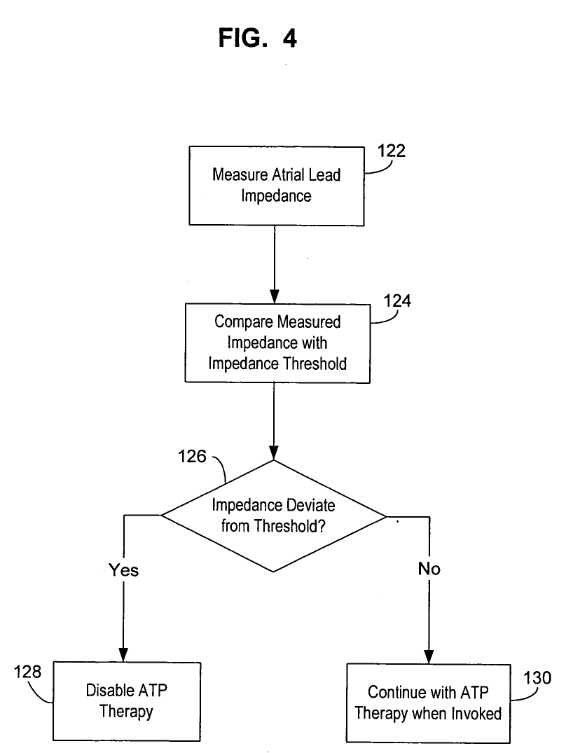

FIG. 4 is a flow diagram depicting several processes of an atrial ATP management methodology implemented in accordance with one particular embodiment of the present invention. According to the methodology shown in FIG. 4, atrial lead impedance is measured 122 for purposes of evaluating atrial lead implantation integrity or atrial lead dislodgement. An atrial lead impedance measurement 122 may be obtained in a number of ways, such as by use of a pace pulse as is known in the art or by use of low energy (e.g., lower than pace pulse energy) stimulation, as will be described in further detail below.

The measured impedance value is compared 124 to an atrial lead impedance threshold that has been developed for the particular patient. If this comparison 126 indicates that the measured impedance value has deviated from (e.g., exceeded) the impedance threshold, dislodgment of the atrial lead is assumed, and ATP therapy is disabled 128. If the comparison 126 does not indicate possible atrial lead dislodgment, ATP therapy is not disabled 130, thereby permitting ATP therapy to be delivered when invoked.

The impedance threshold implicated in blocks 124 and 126 may be developed from a single atrial lead impedance measurement or from several atrial lead impedance measurements. For example, the impedance threshold may be characterized by a mean or a median of several atrial lead impedance measurements. In one approach, the impedance threshold may be characterized by an atrial lead impedance measurement taken immediately before a currently measured impedance. In another approach, the impedance threshold may be characterized by at least one atrial lead impedance measurement taken a predetermined amount of time prior to the impedance measurement. The predetermined amount of time may, for example, be about one day or more than one day.

Measuring the impedance of the atrial lead as implicated in block 122 may involve taking several impedance measurements to characterize the impedance of the atrial lead. In another approach, a single impedance measurement may be used to characterize the impedance of the atrial lead.

According to one lead impedance measuring approach, pacing pulses may be delivered from which impedance measurement values can be derived in a manner known in the art. According to another approach, a stimulus having an energy insufficient to capture the atria is delivered to the subject atrium, and an atrial lead impedance is determined using the delivered stimulus. Use of a low energy stimulus advantageously allows for atrial lead impedance measuring during atrial arrhythmic events or episodes.

In blocks 126 and 128 of FIG. 4, the measured impedance is compared with an impedance threshold developed for the particular patient, and atrial ATP therapy delivery is disabled in response to the measured impedance deviating from the impedance threshold by a predetermined factor. This predetermined factor may be characterized by a percentage change in the measured impedance relative to the impedance threshold, such as a percentage change of at least 25% (e.g., at least a 25% to 50% change). The predetermined factor may also be characterized by a fixed delta change in the measured impedance relative to the impedance threshold, such as a fixed delta change of at least 25% (e.g., at least a 25% to 50% change). According to a further approach, the predetermined factor may be characterized by both a percentage change and a fixed delta change in the measured impedance relative to the impedance threshold.

It is noted that atrial lead impedance tolerances may affect detection of atrial lead impedance changes considered to be sufficiently large as to warrant disablement of atrial ATP therapy delivery. For example, if the atrial lead impedance tolerances are relatively large, such as 20%, it may be difficult to detect actual lead impedance changes from expected variations due to such tolerances. However, because the impedance threshold is established on a per patient basis, any such expected tolerances can be considered by the physician when programming the impedance threshold for detecting large, unexpected changes in atrial lead impedance.

Atrial lead impedance testing at block 122 of FIG. 4 may be performed periodically, such as daily, and/or prior to atrial ATP therapy delivery. For example, atrial lead impedance may be measured after detection of an atrial arrhythmic event and prior to atrial ATP therapy delivery. Additionally or alternatively, atrial lead impedance may be measured after an atrial arrhythmic episode is declared and prior to atrial ATP therapy delivery. In another approach, several atrial lead impedance measurements can be taken after detection of an atrial arrhythmic event or declaring of an atrial arrhythmic episode and prior to atrial ATP therapy delivery.

According to one approach, for example, the most recent daily atrial lead impedance value could be compared to the previous daily atrial lead impedance value. The most recent daily atrial lead impedance value may also be compared to a mean or median of several previous daily lead impedance measurement values. Comparing the most recent daily atrial lead impedance measurement to the previous daily atrial lead impedance represents a relatively conservative approach, but may be too sensitive for disabling atrial ATP therapy in some cases. Comparing the most recent daily atrial lead impedance measurement to a mean or median value may mask large day-to-day impedance changes. Because the impedance threshold is established for a particular patient by the physician, the specific comparison methodology can be determined, refined, and fine-tuned for the particular patient.

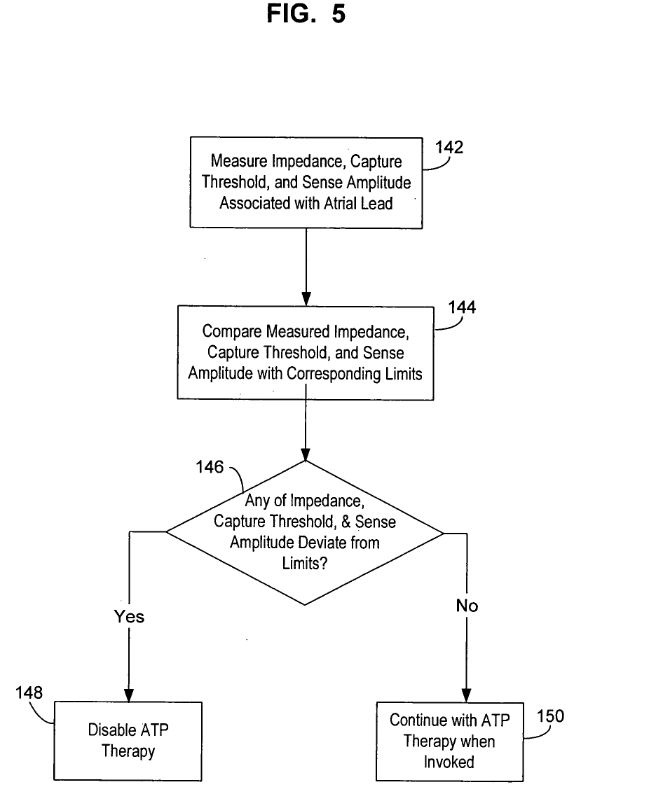

FIG. 5 is a flow diagram depicting several processes of an atrial ATP management methodology implemented in accordance with another embodiment of the present invention. According to the methodology shown in FIG. 5, several atrial lead parameters are measured and processed to enhance detection of atrial lead dislodgement and disablement of atrial ATP therapy in response to same. In this illustrative embodiment, impedance, capture threshold, and sense amplitude associated with an atrial lead are measured for purposes of evaluating atrial lead implantation integrity or atrial lead dislodgement. Although impedance and two other parameters are described in connection with this embodiment, it is understood that two or greater than three atrial lead parameters may be used to enhance detection of atrial lead dislodgement and disablement of atrial ATP therapy.

In block 142, an impedance, a capture threshold, and a sense amplitude respectively associated with an atrial lead for a particular patient are measured. The impedance, capture threshold, and sense amplitude measurements are compared 144 with impedance, capture threshold, and sense amplitude limits, respectively. Atrial ATP therapy delivery is disabled 146, 148 in response to any of the impedance, capture threshold, and sense amplitude measurements deviating from the impedance, capture threshold, and sense amplitude limits by predetermined impedance, capture threshold, and sense amplitude factors, respectively. If none of the impedance, capture threshold, and sense amplitude limits is met or exceeded, atrial ATP therapy remains available 150 when invoked.

The impedance, capture threshold, and sense amplitude limits are selected on a per patient basis to facilitate easy and accurate detection of atrial lead implantation integrity/dislodgment for a particular patient. For example, a percentage change in the capture threshold of at least 25% (e.g., at least a 25% to 50% change) indicates a high likelihood of atrial lead dislodgement. A fixed delta change in the capture threshold of at least 25% (e.g., at least a 25% to 50% change) percent indicates a high likelihood of atrial lead dislodgement. A percentage change in the atrial sense amplitude of at least 25% (e.g., at least a 25% to 50% change) indicates a high likelihood of atrial lead dislodgement. A fixed delta change in the atrial sense amplitude of at least 25% (e.g., at least a 25% to 50% change) indicates a high likelihood of atrial lead dislodgement.

According to this embodiment, detecting an aberrant atrial lead condition indicative of lead dislodgment is based on detection of a large, unexpected change in any one of atrial lead impedance, capture threshold, or atrial sense amplitude. In certain configurations and patients, it may be desirable to require detection of excessively large changes in two of the three measured atrial lead parameters prior to disabling atrial ATP therapy delivery, with lead impedance preferably being one of the two parameters.

It is contemplated that an ambiguity in the impedance, capture threshold, and sense amplitude deviations may be detected. In one approach, atrial ATP therapy delivery is disabled in response to the detected ambiguity. According to another approach in which such an ambiguity is detected, atrial ATP therapy delivery is disabled in response to the measured impedance deviating from the impedance limit by a predetermined factor. In a further approach, atrial ATP therapy delivery is disabled in response to the measured impedance deviating from the impedance limit by the predetermined factor irrespective of the presence or lack of ambiguity relative to the capture threshold and sense amplitude deviations. In these last two approaches, atrial lead impedance is considered the more reliable indicator for detecting atrial lead dislodgment.

An atrial ATP management methodology of the present invention can successfully detect occurrence of atrial lead dislodgement during an atrial arrhythmic event or episode. As was discussed previously, a stimulus having an energy insufficient to capture the atria may be used to determined atrial lead impedance during an atrial arrhythmia.

Capture threshold and atrial sense amplitude, however, are considered unreliable indicators of atrial lead dislodgment during an atrial arrhythmic event or episode. For example, it is common for intrinsic atrial signal amplitudes to decrease during atrial tachyarrhythmias, such that large changes in atrial sense amplitude would be expected. Relying on intrinsic atrial signal amplitude during atrial tachyarrhyhmias could result in false positive detections of lead dislodgement. Because of fast atrial rates associated with atrial tachyarrhythmia, it is not possible to perform atrial pacing threshold tests during an atrial arrhythmic event or episode.

In an atrial lead dislodgement methodology that employs impedance, capture threshold, and sense amplitude parameters, lead impedance is considered the only reliable parameter of these three parameters that can be used to accurately detect atrial lead dislodgement during atrial tachyarrhyhmias. Upon detection of an atrial arrhythmia, atrial ATP therapy is disabled in response to the measured impedance deviating from the impedance limit by a predetermined factor. For example, upon detection of an atrial arrhythmia, ATP therapy delivery is disabled in response only to the measured impedance deviating from the impedance limit by the predetermined factor, and deviations of the capture threshold and sense amplitude from their respective predetermined factors are ignored.

It is noted that the capture threshold and sense amplitude measurements and limits may be developed in a manner discussed above with regard to impedance measurements and limits. For example, capture threshold and sense amplitude limits may be developed from a single atrial lead measurement or from several atrial lead measurements. Capture threshold and sense amplitude limits may be developed from one or more atrial lead measurements taken immediately before presently made capture threshold and sense amplitude measurements, or from one or more atrial lead measurements taken a predetermined amount of time prior to the respective current capture threshold and sense amplitude measurements.

The predetermined capture threshold and sense amplitude factors may be developed in a manner discussed above with regard to the predetermined impedance factor. For example, the predetermined capture threshold and sense amplitude factors may be characterized by a percentage change in the capture threshold and sense amplitude measurements relative to the capture threshold and sense amplitude limits, respectively. The predetermined capture threshold and sense amplitude factors may also be characterized by a fixed delta change in the capture threshold and sense amplitude measurements relative to the capture threshold and sense amplitude limits, respectively. Further, the predetermined capture threshold and sense amplitude factors may be characterized by both a percentage change and a fixed delta change in the capture threshold and sense amplitude measurements relative to the capture threshold and sense amplitude limits, respectively.

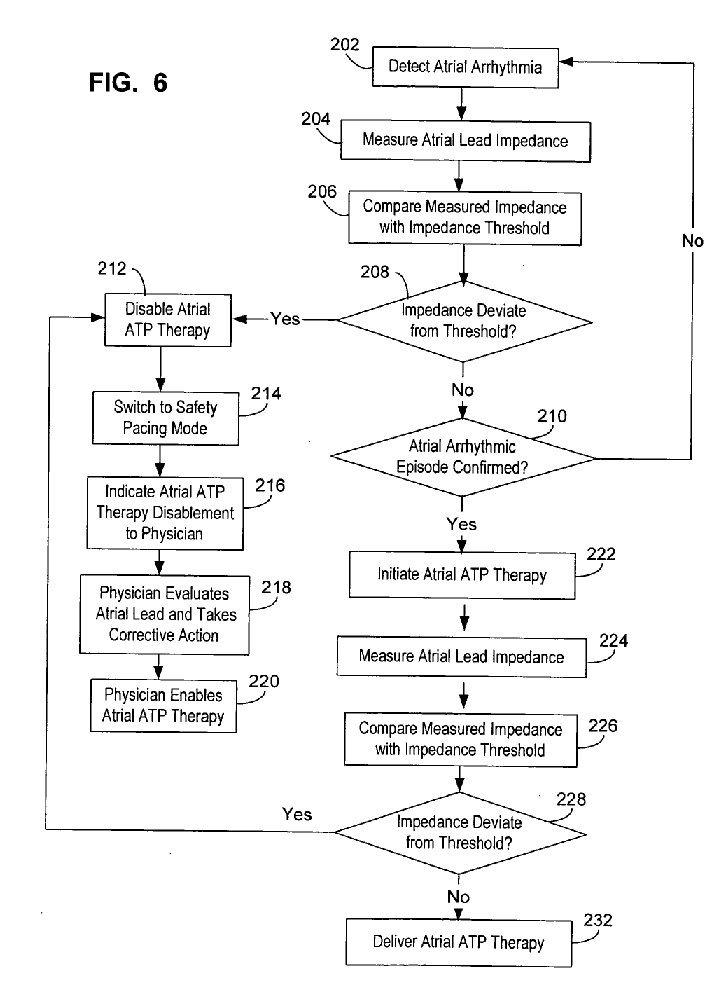

Turning now to FIG. 6, there is shown a flow diagram depicting several processes of an atrial ATP management methodology implemented using a cardiac rhythm management (CRM) device in accordance with an embodiment of the present invention. According to the methodology shown in FIG. 6, an atrial arrhythmia is detected 202 using a known detection technique. Atrial lead impedance is measured 204 in response to detection of the atrial arrhythmia. The measured atrial lead impedance value is compared 206 with an impedance threshold. If the measured impedance value deviates 208 from the impedance threshold by an amount sufficient to indicate probable atrial lead dislodgement, atrial ATP therapy is disabled 212.

If atrial ATP therapy is disabled 212, the CRM device switches 214 to a safe pacing mode, such as a non-atrial tracking mode (e.g., VVI). An indication of atrial ATP therapy disablement, such as a flag set in device memory, is communicated 216 to the patient's physician upon establishing communication between the CRM device and an external programmer, advanced patient management (APM) system or other system capable of communicating with the CRM device. The physician may then evaluate 218 the integrity of the atrial lead and determine if corrective action is required. After completion of the physician's evaluation (e.g., confirming absence of lead dislodgement or replacing/re-positioning a dislodged lead confirmed by the physician), the physician enables atrial ATP therapy delivery.

If the measured impedance value does not deviate 208 from the impedance threshold by an amount sufficient to indicate probable atrial lead dislodgement, the persistence or termination of an atrial arrhythmic episode is confirmed 210. If not confirmed, the processes beginning at block 202 are repeated.

If confirmed, atrial ATP therapy is initiated 222. Prior to delivering atrial ATP therapy 232, one or more additional atrial lead impedance tests are performed. For example, prior to delivering atrial ATP therapy 232, the atrial lead impedance is measured 224 and compared 226 with the predetermined impedance threshold. If the measured impedance deviates 228 from the impedance threshold by an amount sufficient to indicate probable atrial lead dislodgement, atrial ATP therapy is disabled 212, followed by processes 214-220 shown in FIG. 6. If, however, the measured impedance does not deviate 228 from the impedance threshold by an amount sufficient to indicate probable atrial lead dislodgement, atrial ATP therapy is delivered 232.

The CRM device discussed above may have components and functionality previously described with regard to FIG. 2. For example, and with reference to FIG. 2, a CRM device 22 or other implantable pulse generator includes an implantable housing 36. Detection circuitry 104 and energy delivery circuitry 106 are provided in the housing 36. A lead system is coupled to the detection and energy delivery circuitry 104, 106. The lead system includes at least an atrial lead.

A control system 100 is provided in the housing 36 and coupled to memory 102 within which an impedance threshold developed for a particular patient is stored. The control system 100 measures an impedance of an atrial lead for the particular patient and compares the measured impedance with the impedance threshold stored in memory 102. The control system disables atrial ATP therapy delivery in response to the measured impedance deviating from the impedance threshold by a predetermined factor, such as a predetermined factor previously discussed above.

The control system 100 may measure the atrial lead impedance using a pace pulse delivered by the energy delivery circuitry 105 via the atrial lead. The control system may also measure the impedance using a stimulus delivered via the atrial lead, wherein the stimulus has an energy insufficient to effect atrial capture. The control system 100 may measure atrial lead impedance after detection of an atrial arrhythmic event or episode and prior to atrial ATP therapy delivery.

In another embodiment, the control system 100 may further measure a capture threshold and a sense amplitude respectively associated with the atrial lead. The control system 100 compares the capture threshold and sense amplitude measurements with capture threshold and sense amplitude limits, respectively. The control system 100 disables atrial ATP therapy delivery in response to one or more of the impedance measurement deviating from the impedance threshold by a predetermined impedance factor or the capture threshold and sense amplitude measurements deviating from the capture threshold and sense amplitude limits by predetermined capture threshold and sense amplitude factors, respectively. The control system 100 may further detect an ambiguity in the impedance, capture threshold, and sense amplitude deviations, and disable atrial ATP therapy delivery in manners previously described above in response to detecting such an ambiguity.

It will, of course, be understood that various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

1. A method of managing atrial antitachycardia pacing (ATP) therapy in response to possible atrial lead dislodgment, comprising:
measuring an impedance of an atrial lead for a particular patient;
comparing the measured impedance with an impedance threshold developed for the particular patient; and
disabling atrial ATP therapy delivery in response to the measured impedance deviating from the impedance threshold by a predetermined factor.

2. The method according to claim 1, wherein the impedance threshold is developed from a single atrial lead impedance measurement.

3. The method according to claim 1, wherein the impedance threshold is developed from a plurality of atrial lead impedance measurements.

4. The method according to claim 1, wherein the impedance threshold is characterized by a mean or a median of a plurality of atrial lead impedance measurements.

5. The method according to claim 1, wherein the impedance threshold is characterized by an atrial lead impedance measurement taken immediately before a currently measured impedance.

6. The method according to claim 1, wherein the impedance threshold is characterized by at least one atrial lead impedance measurement taken a predetermined amount of time prior to the impedance measurement.

7. The method according to claim 6, wherein the predetermined amount of time is about one day.

8. The method according to claim 6, wherein the predetermined amount of time is more than one day.

9. The method according to claim 1, wherein measuring the impedance of the atrial lead comprises taking a plurality of impedance measurements to characterize the impedance of the atrial lead.

10. The method according to claim 1, wherein measuring the impedance of the atrial lead comprises taking a single impedance measurement to characterize the impedance of the atrial lead.

11. The method according to claim 1, wherein the predetermined factor is characterized by a percentage change in the measured impedance relative to the impedance threshold.

12. The method according to claim 1, wherein the predetermined factor is characterized by a fixed delta change in the measured impedance relative to the impedance threshold.

13. The method according to claim 1, wherein the predetermined factor is characterized by both a percentage change and a fixed delta change in the measured impedance relative to the impedance threshold.

14. The method according to claim 1, wherein measuring the impedance comprises delivering a pace pulse via the atrial lead and deriving the impedance measurement using the delivered pace pulse.

15. The method according to claim 1, wherein measuring the impedance comprises delivering a stimulus via the atrial lead and deriving the impedance measurement using the delivered stimulus, the stimulus having an energy insufficient to effect atrial capture.

16. The method according to claim 1, wherein the impedance is measured after detection of an atrial arrhythmic event and prior to atrial ATP therapy delivery.

17. The method according to claim 1, wherein the impedance is measured after an atrial arrhythmic episode is declared and prior to atrial ATP therapy delivery.

18. The method according to claim 1, wherein measuring the impedance comprises taking a plurality of impedance measurements after detection of an atrial arrhythmic event and prior to atrial ATP therapy delivery.

19. The method according to claim 1, wherein measuring the impedance comprises taking a plurality of impedance measurements after an atrial arrhythmic episode is declared and prior to atrial ATP therapy delivery.

20. A method of managing atrial antitachycardia pacing (ATP) therapy in response to possible atrial lead dislodgment, comprising:
measuring an impedance, a capture threshold, and a sense amplitude respectively associated with an atrial lead for a particular patient;
comparing the impedance, capture threshold, and sense amplitude measurements with impedance, capture threshold, and sense amplitude limits, respectively; and
disabling atrial ATP therapy delivery in response to any of the impedance, capture threshold, and sense amplitude measurements deviating from the impedance, capture threshold, and sense amplitude limits by predetermined impedance, capture threshold, and sense amplitude factors, respectively.

21. The method according to claim 20, further comprising:
detecting an ambiguity in the impedance, capture threshold, and sense amplitude deviations; and
disabling atrial ATP therapy delivery in response to the detected ambiguity.

22. The method according to claim 20, further comprising:
detecting an ambiguity in the impedance, capture threshold, and sense amplitude deviations; and
in response to the detected ambiguity, disabling atrial ATP therapy delivery in response to the measured impedance deviating from the impedance limit by the predetermined factor.

23. The method according to claim 22, further comprising disabling atrial ATP therapy delivery in response to the measured impedance deviating from the impedance limit by the predetermined factor irrespective of a lack of ambiguity relative to the capture threshold and sense amplitude deviations.

24. The method according to claim 20, wherein disabling atrial ATP therapy delivery comprises, upon detection of an atrial arrhythmia, disabling ATP therapy in response to the measured impedance deviating from the impedance limit by the predetermined factor.

25. The method according to claim 20, wherein disabling ATP therapy delivery comprises, upon detection of an atrial arrhythmia, ignoring the capture threshold and sense amplitude-deviations, and disabling ATP therapy in response only to the measured impedance deviating from the impedance limit by the predetermined factor.

26. The method according to claim 20, wherein one or more of the impedance, capture threshold, and sense amplitude limits are developed from a single atrial lead measurement.

27. The method according to claim 20, wherein one or more of the impedance, capture threshold, and sense amplitude limits are developed from a plurality of atrial lead measurements.

28. The method according to claim 20, wherein one or more of the impedance, capture threshold, and sense amplitude limits are developed from one or more atrial lead measurements taken immediately before currently made impedance, capture threshold, and sense amplitude measurements.

29. The method according to claim 20, wherein one or more of the impedance, capture threshold, and sense amplitude limits are developed from one or more atrial lead measurements taken a predetermined amount of time prior to the respective impedance, capture threshold, and sense amplitude measurements.

30. The method according to claim 29, wherein the predetermined amount of time is within about one day.

31. The method according to claim 20, wherein the predetermined impedance, capture threshold, and sense amplitude factors are characterized by a percentage change in the impedance, capture threshold, and sense amplitude measurements relative to the impedance, capture threshold, and sense amplitude limits, respectively.

32. The method according to claim 20, wherein the predetermined impedance, capture threshold, and sense amplitude factors are characterized by a fixed delta change in the impedance, capture threshold, and sense amplitude measurements relative to the impedance, capture threshold, and sense amplitude limits, respectively.

33. The method according to claim 20, wherein the predetermined impedance, capture threshold, and sense amplitude factors are characterized by both a percentage change and a fixed delta change in the impedance, capture threshold, and sense amplitude measurements relative to the impedance, capture threshold, and sense amplitude limits, respectively.

34. The method according to claim 20, wherein the impedance measurement is taken after detection of an atrial arrhythmic event and prior to atrial ATP therapy delivery.

35. The method according to claim 20, wherein the impedance measurement is taken after an atrial arrhythmic episode is declared and prior to atrial ATP therapy delivery.

36. An apparatus for managing atrial antitachycardia pacing (ATP) therapy in response to possible atrial lead dislodgment, comprising:
an implantable housing;
detection circuitry provided in the housing;
energy delivery circuitry provided in the housing;
a lead system respectively coupled to the detection and energy delivery circuitry, the lead system comprising at least an atrial lead; and
a control system provided in the housing and coupled to memory within which an impedance threshold developed for a particular patient is stored, the control system measuring an impedance of the atrial lead for the particular patient and comparing the measured impedance with the impedance threshold, the control system disabling atrial ATP therapy delivery in response to the measured impedance deviating from the impedance threshold by a predetermined factor.

37. The apparatus according to claim 36, wherein the impedance threshold is developed from a single atrial lead impedance measurement.

38. The apparatus according to claim 36, wherein the impedance threshold is developed from a plurality of atrial lead impedance measurements.

39. The apparatus according to claim 36, wherein the impedance threshold is characterized by a mean or a median of a plurality of atrial lead impedance measurements.

40. The apparatus according to claim 36, wherein the impedance threshold is characterized by an atrial lead impedance measurement taken immediately before a currently measured impedance.

41. The apparatus according to claim 36, wherein the impedance threshold is characterized by at least one atrial lead impedance measurement taken a predetermined amount of time prior to the measured impedance.

42. The apparatus according to claim 41, wherein the predetermined amount of time is about one day prior to a day on which the impedance measurement is taken.

43. The apparatus according to claim 41, wherein the predetermined amount of time is defined by more than one day prior to a day on which the impedance measurement is taken.

44. The apparatus according to claim 36, wherein the control system measures the impedance of the atrial lead by taking a plurality of impedance measurements.

45. The apparatus according to claim 36, wherein the control system measures the impedance of the atrial lead by taking a single impedance measurement.

46. The apparatus according to claim 36, wherein the predetermined factor is characterized by a percentage change in the measured impedance relative to the impedance threshold.

47. The apparatus according to claim 36, wherein the predetermined factor is characterized by a fixed delta change in the measured impedance relative to the impedance threshold.

48. The apparatus according to claim 36, wherein the predetermined factor is characterized by both a percentage change and a fixed delta change in the measured impedance relative to the impedance threshold.

49. The apparatus according to claim 36, wherein the control system measures the impedance using a pace pulse delivered via the atrial lead.

50. The apparatus according to claim 36, wherein the control system measures the impedance using a stimulus delivered via the atrial lead, the stimulus having an energy insufficient to effect atrial capture.

51. The apparatus according to claim 36, wherein the control system measures the impedance after detection of an atrial arrhythmic event and prior to atrial ATP therapy delivery.

52. The apparatus according to claim 36, wherein the control system measures the impedance after an atrial arrhythmic episode is declared and prior to atrial ATP therapy delivery.

53. The apparatus according to claim 36, wherein the control system measures the impedance by taking a plurality of impedance measurements after detection of an atrial arrhythmic event and prior to atrial ATP therapy delivery.

54. The apparatus according to claim 36, wherein the control system measures the impedance by taking a plurality of impedance measurements after an atrial arrhythmic episode is declared and prior to atrial ATP therapy delivery.

55. The apparatus according to claim 36, wherein the control system further:
measures a capture threshold and a sense amplitude respectively associated with the atrial lead;
compares the capture threshold and sense amplitude measurements with capture threshold and sense amplitude limits, respectively; and
disables atrial ATP therapy delivery in response to one or more of the impedance measurement deviating from the impedance threshold by a predetermined impedance factor or the capture threshold and sense amplitude measurements deviating from the capture threshold and sense amplitude limits by predetermined capture threshold and sense amplitude factors, respectively.

56. The apparatus according to claim 55, wherein the control system further:
detects an ambiguity in the impedance, capture threshold, and sense amplitude deviations; and
in response to the detected ambiguity, disables atrial ATP therapy delivery.

57. The apparatus according to claim 56, wherein the control system disables atrial ATP therapy delivery in response to the measured impedance deviating from the impedance threshold by the predetermined factor irrespective of a lack of ambiguity relative to the capture threshold and sense amplitude deviations.

58. The apparatus according to claim 55, wherein the control system further:
detects an ambiguity in the impedance, capture threshold, and sense amplitude deviations; and
in response to the detected ambiguity, disables atrial ATP therapy delivery in response to the measured impedance deviating from the impedance threshold by the predetermined factor.

59. The apparatus according to claim 36, wherein the control system, upon detection of an atrial arrhythmia, disables atrial ATP therapy delivery in response to the measured impedance deviating from the impedance threshold by the predetermined factor.

60. The apparatus according to claim 36, wherein the control system, upon detection of an atrial arrhythmia, ignores the capture threshold and sense amplitude deviations and disables atrial ATP therapy in response to only the measured impedance deviating from the impedance threshold by the predetermined factor.

61. A system for managing atrial antitachycardia pacing (ATP) therapy in response to possible atrial lead dislodgment, comprising:
means for measuring an impedance of an atrial lead for a particular patient;
means for comparing the measured impedance with an impedance threshold developed for the particular patient; and
means for disabling atrial ATP therapy delivery in response to the measured impedance deviating from the impedance threshold by a predetermined factor.

62. A system for managing atrial antitachycardia pacing (ATP) therapy in response to possible atrial lead dislodgment, comprising:
means for measuring an impedance associated with an atrial lead for a particular patient;
means for measuring a capture threshold associated with the atrial lead for the particular patient;
means for measuring a sense amplitude associated with the atrial lead for the particular patient;
means for comparing impedance, capture threshold, and sense amplitude measurements with impedance, capture threshold, and sense amplitude limits, respectively; and
means for disabling atrial ATP therapy delivery in response to any of the impedance, capture threshold, and sense amplitude measurements deviating from the impedance, capture threshold, and sense amplitude limits by predetermined impedance, capture threshold, and sense amplitude factors, respectively.
